SEQUENCE LISTING (1 sequence)

SEQ ID NO: 1 - protein, 546 aa, Varicella zoster
MGTVNKPVVGVLMGFGIITGTLRITNPVRASVLRYDDFHIDEDKLDTNSVYEPYYHSDHAESSWVNRGESSRKAYDHNSPYIWPRNDYDGFLENAHEHHGVYNQGRGIDSGERLMQPTQMSAQEDLGDDTGIHVIPTLNGDDRHKIVNVDQRQYGDVFKGDLNPKPQGQRLIEVSVEENHPFTLRAPIQRIYGVRYTETWSFLPSLTCTGDAAPAIQHICLKHTTCFQDVVVDVDCAENTKEDQLAEISYRFQGKKEADQPWIVVNTSTLFDELELDPPEIEPGVLKVLRTEKQYLGVYIWNMRGSDGTSTYATFLVTWKGDEKTRNPTPAVTPQPRGAEFHMWNYHSHVFSVGDTFSLAMHLQYKIHEAPFDLLLEWLYVPIDPTCQPMRLYSTCLYHPNAPQCLSHMNSGCTFTSPHLAQRVASTVYQNCEHADNYTAYCLGISHMEPSFGLILHDGGTTLKFVDTPESLSGLYVFVVYFNGHVEAVAYTVVSTVDHFVNAIEERGFPPTAGQPPATTKPKEITPVNPGTSPLIRYAAWTGGLA

(19) United States
(12) Patent Application Publication
(10) Pub. No.: US 2018/0008700 A1
(43) Pub. Date: Jan. 11, 2018

(54) VACCINATION

(71) Applicant: GLAXOSMITHKLINE BIOLOGICALS S.A., Rixensart (BE)

(72) Inventors: Thomas Charles HEINEMAN, King of Prussia, PA (US); Edouard Guy Christian LEDENT, Rixensart (BE); Nicolas Pierre Fernand LECRENIER, Wavre (BE)

(73) Assignee: GLAXOSMITHKLINE BIOLOGICALS S.A., Rixensart (BE)

(21) Appl. No.: 15/535,723

(22) PCT Filed: Dec. 16, 2015
(86) PCT No.: PCT/EP2015/079967

§ 371 Date: Jun. 14, 2017

(30) Foreign Application Priority Data
Dec. 18, 2014 (GB) 1422686.4
Feb. 26, 2015 (GB) 1503266.7
Oct. 26, 2015 (GB) 1518917.8

(51) Int. Cl. A61K 39/25; A61K 39/39; C12N 7/00; A61K 39/00

(52) U.S. Cl. CPC A61K 39/25; C12N 7/00; A61K 39/39; A61K 2039/55555; A61K 2039/55; A61K 2039/545; A61K 2039/55577; C12N 2710/16734; A61K 2039/55572

(57) ABSTRACT

The present invention relates to compositions for use in and methods for protecting against Herpes Zoster (HZ).

### TECHNICAL FIELD

The present invention relates to methods for inducing sustained protection against and prevention of Herpes Zoster or post herpetic neuralgia with high efficacy, in particular in elderly and immunocompromised human patients.

### BACKGROUND

Herpes Zoster (HZ), also known as shingles, is a common and often debilitating disease that occurs primarily in older or immunocompromised individuals. HZ is caused by the symptomatic reactivation of latent varicella zoster virus (VZV) in the dorsal root and cranial ganglia. The virus is usually acquired during childhood as chickenpox.

The only vaccine currently available with demonstrated efficacy against HZ or post herpetic neuralgia is a live attenuated vaccine of VZV OKA strain, marketed as Zostavax™. In the overall population 60 YOA), Zostavax™ reduced the incidence of HZ by 51.3% (p-value <0.001), although its effectiveness decreased with the age of the vaccine. In particular, vaccine efficacy (VE) diminished to 37.6% among persons in older age groups (≧70 years of age). Zostavax™ is contraindicated in persons with immunodeficiency due to malignancy, human immunodeficiency virus (HIV) infection or immunosuppressive medical therapy. (Zostavax™ EMA SPC 2012; Oxman et al. N Engl J Med 2005; 352:2271-2284; Schmader et al. Clin. Infect. Diseases 2012 April; 54(7):922-8). Morrison V. A. et al. reported on the decline in efficacy of Zostavax™ becoming increasingly limited beyond 5-8 years post-vaccination and to be no longer statistically significant beyond 8 years (Morrison et al. Clin. Infect. Diseases advance access publication Nov. 20, 2014).

An adjuvanted subunit VZV immunogenic composition is described in WO2006/094756 (U.S. Pat. No. 7,939,084, which is incorporated herein by reference). Leroux-Roels I. et al. (J. Infect. Diseases 2012:206 1280-1290) report on a phase I/II clinical trial of the adjuvanted VZV gE subunit vaccine evaluating safety and immunogenicity. To date no immunological correlate of protection has been identified.

There is still an ongoing need in the art for HZ vaccination with high efficacy throughout populations at risk and/or with a sustained protection profile, as well as a favourable safety profile.

### SUMMARY OF THE INVENTION

The present invention relates to compositions for use in and methods for protecting against HZ resulting in an unprecedented efficacy and duration of protection with a minimal number of vaccine administrations.

In addition, following the immunisation of human individuals against HZ or post herpetic neuralgia (PHN) using the immunogenic, e.g. vaccine, composition, efficacy does not decline with age and remains exceptionally high at older age. Furthermore, the high levels of efficacy remain persistently high years after immunisation. It will be apparent to one of ordinary skill in the art that efficacy is assessed in a population of individuals treated; efficacy and duration of efficacy will vary among individuals vaccinated.

The invention thus relates to an immunogenic, e.g. vaccine, composition comprising a VZV gE antigen truncated to remove the carboxy terminal anchor region, in combination with an adjuvant comprising a saponin, a TLR-4 agonist and liposomes for use in a method for protection against or prevention of herpes zoster (HZ) and/or post herpetic neuralgia for at least 4 years post-vaccination.

The invention further relates to an immunogenic, e.g. vaccine, composition comprising a VZV gE antigen truncated to remove the carboxy terminal anchor region, in combination with an adjuvant comprising a saponin, a TLR-4 agonist and liposomes for use in a method for protection against or prevention of herpes zoster (HZ) and/or post herpetic neuralgia comprising the step of administering 2 doses of the immunogenic, e.g. vaccine, composition to an individual older than 70 years of age.

The invention also relates to a method for protection against or prevention of herpes zoster (HZ) and/or post herpetic neuralgia for at least 4 years post-vaccination comprising the steps of administering to a human individual an immunogenic, e.g. vaccine, composition comprising a VZV gE antigen truncated to remove the carboxy terminal anchor region, in combination with an adjuvant comprising a saponin, a TLR-4 agonist and liposomes.

The invention also relates to a method for protection against or prevention of herpes zoster (HZ) and/or post herpetic neuralgia comprising the steps of administering 2 doses of an immunogenic, e.g. vaccine, composition comprising a VZV gE antigen truncated to remove the carboxy terminal anchor region, in combination with an adjuvant comprising a saponin, a TLR-4 agonist and liposomes to a human individual older than 70 years of age.

The invention also relates to a method for protecting against, preventing or reducing the incidence of herpes zoster and/or post herpetic neuralgia in an individual comprising the steps of:

a. selecting a subject from a population that is poorly protected by a live attenuated VZV composition, and,
b. administering a first and a second dose of an immunogenic, e.g. vaccine, composition comprising a VZV gE antigen truncated to remove the carboxy terminal anchor region, in combination with an adjuvant comprising a saponin, a TLR-4 agonist and liposomes, wherein the protection, prevention or reduction of incidence of HZ and/or PHN lasts for at least 4 years after administration of the second dose.

The invention also relates to a method for protecting against, preventing or reducing the incidence of herpes zoster and/or post herpetic neuralgia in an individual comprising the steps of:

a. selecting a subject from a population consisting of individuals older than 70 years of age and/or immunocompromised individuals, and,
b. administering a first and a second dose of an immunogenic, e.g. vaccine, composition comprising a VZV gE antigen truncated to remove the carboxy terminal anchor region, in combination with an adjuvant comprising a saponin, a TLR-4 agonist and liposomes.

### DESCRIPTION OF THE DRAWINGS

FIG. 1 schematically represents the study design of the clinical trial described in Example 1.

FIG. 2 schematically represents the study design of the clinical trial described in Example 2.

FIG. 3 schematically represents the study design of the clinical trial described in Example 3.

### DETAILED DESCRIPTION

The immunogenic, e.g. vaccine, composition in accordance of the invention comprises a recombinant VZV gE antigen in combination with an adjuvant.

As disclosed herein, a suitable VZV gE antigen is the VZV glycoprotein gE (also known as gp1) or an immunogenic variant thereof, truncated to remove the carboxy terminal anchor region. The complete varicella-zoster virus (VZV) nucleotide sequence was disclosed by Davison et al. (J Gen Virol, 67:1759-1816 (1986)). The wild type or full length gE protein consists of 623 amino acids comprising a signal peptide, the main part of the protein, a hydrophobic anchor region (residues 546-558) and a C-terminal tail. In one aspect, a VZV gE C-terminal truncate (also referred to truncated gE or gE truncate) is used whereby the truncation removes 4 to 20 percent of the total amino acid residues from the carboxy terminal end, e.g. lacking residues 547 to 623. In an alternative embodiment, the truncated gE lacks the carboxy terminal anchor region (e.g. by an internal deletion in the C-terminal region, suitably approximately amino acids 547-558 of the wild type sequence). In one embodiment, VZV gE antigen is a truncated gE comprising or consisting of the sequence of SEQ ID NO. 1. In a further embodiment, the VZV gE antigen is not presented in the form of a fusion protein comprising a further (non-gE) VZV protein or immunologically active fragment thereof.

The VZV gE antigen, including anchorless VZV gE antigens (which are also immunogenic variants) and production thereof are described in EP0405867 (incorporated herein by reference) and references therein [see also Vafai A. Antibody binding sites on truncated forms of varicalla-zoster virus gpl(gE) glycoprotein Vaccine 1994 12:1265-9]. EP0192902 also discloses gE and production thereof. Truncated gE is also disclosed by Haumont et al. Virus Research (1996) vol 40, p 199-204, herein incorporated fully by reference. An adjuvanted VZV gE composition suitable for use in accordance of the present invention is disclosed in WO2006/094756 (U.S. Pat. No. 7,939,084, which is incorporated herein by reference), i.e. a carboxy terminally truncated VZV gE in combination with an adjuvant comprising QS21, 3D-MPL and liposomes further containing cholesterol. Leroux-Roels I. et al. (J. Infect. Diseases 2012:206 1280-1290) reported on a phase I/II clinical trial evaluating the adjuvanted VZV truncated gE subunit vaccine.

As used herein the term “variant” refers to an antigen that is modified relative to its naturally occurring form. As disclosed herein, a suitable “variant” is an “immunogenic variant”, thus which is sufficiently similar to native antigens to retain antigenic properties and remains capable of inducing an immune response which is cross-reactive with the native antigen. A variant polypeptide may contain a number of substitutions, preferably conservative substitutions, i.e. a substitution of one amino acid by another one with similar properties such as the aliphatic amino acids Val, Ile, Leu, Met or basic amino acids Lys, Arg, His or aromatic amino acids Phe, Tyr, Trp, (for example, 1-50, such as 1-25, in particular 1-10, or 1, 2, 3, 4, 5, 6, 7, 8, 9 or 10 alterations, and especially 1 amino acid residue(s) may be altered, e.g. substituted or deleted) when compared to the reference sequence, i.e. wild type sequence. In particular, variants with respect to SEQ ID No. 1 are contemplated. Suitably such substitutions do not occur in the region of a major epitope (e.g. immunologically important epitope), and do not therefore have a significant impact on the immunogenic properties of the antigen. VZV gE is known to contain B cell and CD4+ T cell epitopes as disclosed by R. E. Bergen et al. (Viral Immunology, 4 (3) (1991), pp. 151-166), W. J. Fowler et al. (Virology, 214 (2) (1995), pp. 531-540), G. N. Malavige et al. (Clin Exp Immunol, 152 (3) (2008), pp. 522-531) and L. Wu & B. Forghani (Arch Virol, 142 (2) (1997), pp. 349-362). Protein variants may also include those wherein additional amino acids are inserted compared to the reference sequence, for example, such insertions may occur at 1-10 locations (such as 1-5 locations, suitably 1 or 2 locations, in particular 1 location) and may, for example, involve the addition of 50 or fewer amino acids at each location (such as 20 or fewer, in particular 10 or fewer, especially 5 or fewer). Suitably such insertions do not occur in the region of an epitope, and do not therefore have a significant impact on the immunogenic properties of the antigen. One example of insertions includes a short stretch of histidine residues (e.g. 2-6 residues) to aid expression and/or purification of the antigen in question. Variants also include those wherein amino acids have been deleted compared to the reference sequence, for example, such deletions may occur at 1-10 locations (such as 1-5 locations, suitably 1 or 2 locations, in particular 1 location) and may, for example, involve the deletion of 20 or fewer amino acids at each location (such as 10 or fewer, in particular 5 or fewer, especially 2 or fewer). Suitably such deletions do not occur in the region of an epitope, and do not therefore have a significant impact on the immunogenic properties of the antigen. The skilled person will recognise that a particular protein variant may comprise substitutions, deletions and additions (or any combination thereof). Variants preferably exhibit at least about 70% identity, more preferably at least about 80% identity and most preferably at least about 90% identity (such as at least about 95%, at least about 98% or at least about 99%) to the associated reference sequence. Examples of algorithms that are suitable for determining percent sequence identity and sequence similarity are the BLAST and BLAST 2.0 algorithms, which are described in Altschul et al., Nuc. Acids Res. 25:3389-3402 (1977) and Altschul et al., J. Mol. Biol. 215:403-410 (1990), respectively. Whether or not a given variant raises such an immune response, may be measured by a suitable immunological assay such as an ELISA or flow cytometry.

The amount of VZV gE antigen used in the immunisation of human individuals against HZ or PHN is selected as an amount which induces an immunoprotective response without significant, adverse side effects in typical vaccines. Such amount will vary depending upon which specific antigen is employed and how it is presented. Generally, it is expected that each dose will comprise 1-1000 μg of protein, such as 2-100 μg, or 5-60 μg. Where VZV gE antigen is used then in one aspect 25-100 μg of gE may be used in humans, such as 40-100 μg of gE for human use, in one aspect about 25 μg, about 50 μg or about 100 μg of gE, suitably 25 μg, 50 μg or 100 μg of gE. In a preferred embodiment, VZV gE antigen (e.g. of SEQ ID NO. 1) is used in a 50 μg dose. As disclosed herein, “dose” is the amount administered in a single administration.

As disclosed herein, a suitable adjuvant comprises a TLR-4 ligand, and a saponin in a liposomal formulation.

A particularly suitable saponin for use in the present invention is Quil A and its derivatives. Quil A is a saponin preparation isolated from the South American tree Quillaja Saponaria Molina and was first described by Dalsgaard et al. in 1974 (“Saponin adjuvants”, Archiv. für die gesamte Virusforschung, Vol. 44, Springer Verlag, Berlin, p 243-254) to have adjuvant activity. Purified fractions of Quil A have been isolated by HPLC which retain adjuvant activity without the toxicity associated with Quil A (EP 0 362 278), for example QS7 and QS21 (also known as QA7 and QA21). QS21 is a natural saponin derived from the bark of Quillaja saponaria Molina, which typically induces CD8+ cytotoxic T cells (CTLs), Th1 cells and a predominant IgG2a antibody response and is a preferred saponin in the context of the present invention.

Suitably, the saponin is provided in its less reactogenic composition where it is quenched with an exogenous sterol. Suitable sterols include β-sitosterol, stigmasterol, ergosterol, ergocalciferol and cholesterol. In one particular embodiment, the adjuvant composition comprises cholesterol as sterol. These sterols are well known in the art, for example cholesterol is disclosed in the Merck Index, 11th Edn., page 341, as a naturally occurring sterol found in animal fat. Several particular forms of less reactogenic compositions wherein QS21 is quenched with an exogenous cholesterol exist. The saponin/sterol is formulated in a liposomal formulation structure. Methods for obtaining saponin/sterol in a liposomal formulation are described in WO 96/33739 (U.S. Pat. No. 6,846,489, incorporated herein by reference), in particular Example 1. The relative amount of sterol to phospholipid is 1-50% (mol/mol), suitably 20-25%.

Where the active saponin fraction is QS21, the ratio of QS21:sterol will typically be in the order of 1:100 to 1:1 (w/w), suitably between 1:10 to 1:1 (w/w), and preferably 1:5 to 1:1 (w/w). Suitably excess sterol is present, the ratio of QS21:sterol being at least 1:2 (w/w). In one embodiment, the ratio of QS21:sterol is 1:5 (w/w). The sterol is suitably cholesterol.

The adjuvant composition comprises a TLR-4 agonist. A suitable example of a TLR-4 agonist is a lipopolysaccharide, suitably a non-toxic derivative of lipid A, particularly monophosphoryl lipid A or more particularly 3-Deacylated monophoshoryl lipid A (3D-MPL).

3D-MPL is sold under the name MPL by GlaxoSmithKline Biologicals S. A. and is referred throughout the document as MPL or 3D-MPL. See, for example, U.S. Pat. Nos. 4,436,727; 4,877,611; 4,866,034 and 4,912,094 (each of which incorporated herein by reference). 3D-MPL primarily promotes CD4+ T cell responses with an IFN-g (Th1) phenotype. 3D-MPL can be produced according to the methods disclosed in GB 2 220 211 A. Chemically it is a mixture of 3-deacylated monophosphoryl lipid A with 4, 5 or 6 acylated chains. In the compositions of the present invention small particle 3D-MPL may be used to prepare the adjuvant composition. Small particle 3D-MPL has a particle size such that it may be sterile-filtered through a 0.22 μm filter. Such preparations are described in WO 94/21292. Preferably, powdered 3D-MPL is used to prepare the adjuvant compositions of the present invention.

Other TLR-4 agonists which can be used are alkyl Glucosaminide phosphates (AGPs) such as those disclosed in WO98/50399 or U.S. Pat. No. 6,303,347 (processes for preparation of AGPs are also disclosed), suitably RC527 or RC529 or pharmaceutically acceptable salts of AGPs as disclosed in U.S. Pat. No. 6,764,840. Some AGPs are TLR-4 agonists, and some are TLR-4 antagonists. In the present invention, the use of a TLR-4 agonist is contemplated.

Other suitable TLR-4 ligands are as described in WO2003/011223 (US20020176861) and in WO 2003/099195 (U.S. Pat. No. 7,833,993), both incorporated herein by reference, such as compound I, compound II and compound III disclosed on pages 4-5 of WO2003/011223 or on pages 3-4 of WO2003/099195 and in particular those compounds disclosed in WO2003/011223 as ER803022, ER803058, ER803732, ER804053, ER804057m ER804058, ER804059, ER804442, ER804680 and ER804764. For example, one suitable TLR-4 ligand is ER804057.

The adjuvant composition comprises both saponin and a TLR4 agonist. In a specific example, the adjuvant composition comprises QS21 and 3D-MPL.

A TLR-4 agonist such as a lipopolysaccharide, such as 3D-MPL, can be used at amounts of between 1 and 100 μg per human dose of the adjuvant composition. 3D-MPL may be used at a level of about 50 μg, for example between 40-60 μg, suitably between 45-55 μg or between 49 and 51 μg or 50 μg. In a further embodiment, the human dose of the adjuvant composition comprises 3D-MPL at a level of about 25 μg, for example between 20-30 μg, suitable between 21-29 μg or between 22-28 μg or between 23 and 27 μg or between 24 and 26 μg, or 25 μg.

A saponin, such as QS21, can be used at amounts between 1 and 100 μg per human dose of the adjuvant composition. QS21 may be used at a level of about 50 μg, for example between 40-60 μg, suitably between 45-55 μg or between 49 and 51 μg or 50 μg. In a further embodiment, the human dose of the adjuvant composition comprises QS21 at a level of about 25 μg, for example between 20-30 μg, suitable between 21-29 μg or between 22-28 μg or between 23 and 27 μg or between 24 and 26 μg, or 25 μg. QS21 may be present at a dose 60 μg, 55 μg or 30 μg per dose. QS21 may be present in a dose 20 μg, 40 μg or 45 μg per dose.

The weight ratio of TLR-4 agonist to saponin is suitably between 1:5 and 5:1, suitably 1:1. For example, where 3D-MPL is present at an amount of 50 μg or 25 μg, then suitably QS21 may also be present at an amount of 50 μg or 25 μg per human dose of the adjuvant composition.

By “liposomal formulation” is meant that the saponin and TLR-4 agonist are formulated with liposomes. The liposomes intended for the present invention contain a neutral lipid, for example phosphatidylcholine, which is suitably non-crystalline at room temperature, for example eggyolk phosphatidylcholine, dioleoyl phosphatidylcholine (DOPC) or dilauryl phosphatidylcholine. In a preferred embodiment, the liposomes of the present invention contain DOPC. The liposomes may also contain a charged lipid which increases the stability of the lipsome-QS21 structure for liposomes composed of saturated lipids. In these cases the amount of charged lipid is suitably 1-20% w/w, preferably 5-10%.

WO2013/041572 (US20140234403, incorporated herein by reference), in particular examples 3 and 4, further discloses methods for making a liposome bulk preparation of DOPC liposomes further containing cholesterol and 3D-MPL, for further mixing with QS21, thereby obtaining an adjuvant suitable for use in accordance with the present invention.

The immunisation schedule may comprise several doses of the vaccine composition. In one embodiment of the invention, at least 2 doses of the immunogenic, e.g. vaccine, composition are administered to the individual. In another embodiment, the vaccination consists of 2 doses of the vaccine composition, i.e. following an initial 2 dose vaccination, the individual does not receive further administrations of the immunogenic, e.g. vaccine, composition for at least 3, 4, 5, 6, 7, 8, 9, 10, etc. years. The composition is typically administered via the intramuscular route, although alternative routes may be considered, e.g. intradermal or subcutaneous.

The interval in between administration of several (or 2) doses of the vaccine may be varied between 1 week and about one year (i.e. 12 months), or between 1 month and one year, or between 1 and 3 months, or between 2 and 12 months, or between 2 and 6 months. In one embodiment the interval is 2, 6 or 12 months. Particularly, the interval is 2 months. Also particularly, the interval is 12 months. Alternatively, the interval is 1 year. 1 month interval will typically be within 30 to 48 days. 2 months interval will typically be within 49 to 83 days. 12 month interval will typically be within 335 and 395 days.

The immunogenic, e.g. vaccine, composition in accordance with the invention is for use in vaccination, namely the protection against or prevention of herpes zoster (HZ), i.e. prevention of reactivation of VZV, also referred to as shingles, and/or post herpetic neuralgia (PHN) of a human individual. In one embodiment, the immunogenic, e.g. vaccine, composition is used in the protection against or the prevention of the incidence of herpes zoster. Where HZ does occur then the severity of shingles is suitably reduced compared to an unvaccinated individual (i.e. amelioration of HZ). Also, when HZ does occur, other disease syndromes may develop such as post herpetic neuralgia.

PHN is the most common severe complication of HZ. PHN is defined as pain that persists after the resolution of the HZ rash. Affected patients typically report burning, throbbing, intermittent sharp or electric shock-like pain, or allodynia. Older age is a clear risk factor for PHN. Other risk factors may include a severe HZ rash and a painful HZ prodrome. PHN tends to improve over a period of months. About 70-80% of cases resolve within 1 year, however, in some persons PHN persists for many years (Dworkin et al. 2007. Clin. Infec. Dis.; 44 Suppl. 1: S1-S26). PHN is commonly defined as pain 90 days after rash onset. The intensity, character and duration of PHN vary widely among individuals. Accordingly, a specific questionnaire aimed at evaluating the pain (in terms of magnitude and duration) and discomfort associated with HZ has been specifically designed, called the Zoster Brief Pain Inventory (ZBPI). A copy of said ZBPI questionnaire is available for example in Coplan et al. 2004. J. Pain. 5(6):344-356. This ZBPI is particularly useful, and is routinely used, when assessing, for example in clinical trials, compounds aimed at preventing or protecting against HZ-associated pain, including PHN.

In a further embodiment, the invention relates to the use in the protection against or the prevention of the incidence of post herpetic neuralgia. Where PHN does occur then the severity of the PHN is suitably reduced compared to an unvaccinated individual (i.e. amelioration of PHN). The use or method as disclosed herein will boost an immune response typically induced by natural infection. As disclosed herein, it is understood that prevention of or protection against HZ and/or PHN occurs when the incidence and/or severity of the occurrence of HZ and/or PHN is reduced. Reduction of severity means reduction of overall disease, or any of the clinical manifestations associated with HZ and/or PHN. For example, reduction of severity means a reduction of the pain associated with HZ and/or PHN, which pain can be suitably measured and monitored using the ZBPI questionnaire.

In further embodiments, the use or method according to the invention is to protect against or prevent both HZ and PHN.

The use or method of protecting in accordance with the present invention (the vaccination) provides exceptionally high efficacies. In one embodiment, the efficacy of the vaccination is expressed as the reduction of the occurrence of HZ in a population after receiving the immunogenic, e.g. vaccine, composition compared to placebo. The vaccination efficacy of reducing the occurrence of HZ in a population compared to placebo is 60% or more, suitably 70% or more, suitably 80% or more, suitably 85% or more, suitably 87% or more suitably 90% or more, suitably 95% or more, or, even 97% or more. In particular embodiments, the efficacy is 80% or more, the efficacy is 85% or more, or, the efficacy is 90% or more. In specific embodiments, the efficacy is 80% or more, or, 90% or more.

In another embodiment, the efficacy is expressed as the reduction in the occurrence of post herpetic neuralgia in a population after receiving the immunogenic, e.g. vaccine, composition compared to placebo. The vaccine efficacy of reducing the occurrence of PHN in a population compared to placebo is 70% or more, suitably 80% or more, suitably 85% or more, suitably 87% or more, suitably 90% or more, suitably 95% or more, or, suitably 97% or more. In particular embodiments, the vaccine efficacy of reducing the occurrence of PHN is 80% or more, the efficacy is 85% or more, or, 87% or more, especially in a target population of human individuals 50 years of age or older, 60 years of age or older, 70 years of age or older, or, older than 70 years of age. In another particular embodiment, the efficacy in reducing the occurrence of PHN in individuals 80 years of age or older is 70% or more.

Furthermore, it has surprisingly been found that the efficacy in accordance with the present invention is exceptionally high in all target populations. Contrary to the usual decrease in vaccine efficacy observed in subjects with a waning immune system, efficacy of vaccination using the immunogenic, e.g. vaccine, composition in accordance with the present invention is exceptionally high in all target populations, even in individuals above or older than 70 years of age and/or immune-compromised individuals.

Particular target populations considered in accordance with the present invention are human individuals ≧50 years of age, ≧60 years of age, ≧70 years of age, between 50 and 59 years of age, or, between 60 and 69 years of age; and more in particular are considered subjects that are ≧70 years of age, such as ≧71 years of age, e.g. ≧72 years of age, such as ≧73 years of age, e.g. ≧74 years of age, such as ≧75 years of age, e.g. ≧80 years of age or ≧81 years of age. In a particular embodiment, the target population comprises human individuals older than 70 years of age.

Further particular populations are immune-compromised populations or individuals, such as HIV positive patients or patients suffering from AIDS, transplant patients e.g. renal transplant patients or haematopoietic cell transplant patients, patients suffering heamotological malignancies, solid tumor patients or patients otherwise suffering or at risk of suffering from an acquired immune deficiency e.g. by receiving or being enrolled to receive immunosuppressant therapy, such as chemotherapy or radiotherapy.

In addition, it has surprisingly been found that the efficacy in accordance with the present invention is sustained through the course of at least 3, 4, 5, 6, 7, 8, 9, 10, 11, 12, 13, 14, 15 years post-vaccination, i.e. following the last administration of a dose of the immunogenic, e.g. vaccine, composition administered to the individual, such that e.g. HZ and/or PHN is prevented or the incidence or severity is reduced for at least 3, 4, 5, 6, 7, 8, 9, 10, 11, 12, 13, 14, 15 years post-vaccination. In one embodiment, protection or prevention is provided for at least 5 years post-vaccination. In another embodiment, protection or prevention is provided for at least 8 years post-vaccination. In yet another embodiment, protection or prevention is provided for at least 10 years post-vaccination.

It has in particular been found that the high levels of efficacy are sustained through the at least 3, 4, 5, 6, 7, 8, 9, 10, 11, 12, 13, 14, 15 years post-vaccination, i.e. after administration of the second dose of the immunogenic, e.g. vaccine, composition.

Preferred embodiments of the invention include:
An immunogenic, e.g. vaccine, composition comprising a VZV gE antigen truncated to remove the carboxy terminal anchor region or derivative thereof, in combination with an adjuvant comprising a QS21, a 3D-MPL and liposomes comprising cholesterol for use in a method for protecting against or preventing herpes zoster (HZ) and/or post herpetic neuralgia in individuals 70 years of age or older for at least 5 years.
An immunogenic, e.g. vaccine, composition comprising a VZV gE antigen truncated to remove the carboxy terminal anchor region or derivative thereof, in combination with an adjuvant comprising a QS21, a 3D-MPL and liposomes comprising cholesterol for use in a method for protecting against or preventing herpes zoster (HZ) and/or post herpetic neuralgia in individuals older than 70 years of age for at least 5 years.
An immunogenic, e.g. vaccine, composition comprising a VZV gE antigen truncated to remove the carboxy terminal anchor region or derivative thereof, in combination with an adjuvant comprising a QS21, a 3D-MPL and liposomes comprising cholesterol for use in a method for protecting against or preventing herpes zoster (HZ) and/or post herpetic neuralgia in immune-compromised individuals for at least 5 years.
An immunogenic, e.g. vaccine, composition comprising a VZV gE antigen truncated to remove the carboxy terminal anchor region or derivative thereof, in combination with an adjuvant comprising a QS21, a 3D-MPL and liposomes comprising cholesterol for use in a method for protecting against or preventing herpes zoster (HZ) and/or post herpetic neuralgia in individuals 50 years of age or older reducing the incidence of HZ by at least 80% for at least 5 years.
An immunogenic, e.g. vaccine, composition comprising a VZV gE antigen truncated to remove the carboxy terminal anchor region or derivative thereof, in combination with an adjuvant comprising a QS21, a 3D-MPL and liposomes comprising cholesterol for use in a method for protecting against or preventing herpes zoster (HZ) and/or post herpetic neuralgia in individuals 50 years of age or older reducing the incidence of PHN by at least 80% for at least 5 years.
An immunogenic, e.g. vaccine, composition comprising a VZV gE antigen truncated to remove the carboxy terminal anchor region or derivative thereof, in combination with an adjuvant comprising a QS21, a 3D-MPL and liposomes comprising cholesterol for use in a method for protecting against or preventing herpes zoster (HZ) and/or post herpetic neuralgia in individuals older than 70 years of age reducing the incidence of HZ by at least 80% for at least 5 years.
An immunogenic, e.g. vaccine, composition comprising a VZV gE antigen truncated to remove the carboxy terminal anchor region or derivative thereof, in combination with an adjuvant comprising a QS21, a 3D-MPL and liposomes comprising cholesterol for use in a method for protecting against or preventing herpes zoster (HZ) and/or post herpetic neuralgia in individuals older than 70 years of age reducing the incidence of PHN by at least 80% for at least 5 years.
An immunogenic, e.g. vaccine, composition comprising a VZV gE antigen truncated to remove the carboxy terminal anchor region or derivative thereof, in combination with an adjuvant comprising a QS21, a 3D-MPL and liposomes comprising cholesterol for use in a method for protecting against or preventing herpes zoster (HZ) and/or post herpetic neuralgia in immune-compromised individuals reducing the incidence of HZ by at least 80% for at least 5 years.
An immunogenic, e.g. vaccine, composition comprising a VZV gE antigen truncated to remove the carboxy terminal anchor region or derivative thereof, in combination with an adjuvant comprising a QS21, a 3D-MPL and liposomes comprising cholesterol for use in a method for protecting against or preventing herpes zoster (HZ) and/or post herpetic neuralgia in immune-compromised individuals reducing the incidence of PHN by at least 80% for at least 5 years.

Suitable derivatives are variants as defined herein above. Particularly preferred are the foregoing preferred embodiments wherein the method provides protection for at least 8 years post-vaccination. More particularly preferred are the foregoing preferred embodiments wherein the method provides protection for at least 10 years post-vaccination.

Also particularly preferred are the foregoing preferred embodiments wherein the method reduces the incidence of HZ by at least 90%. More particularly preferred are the foregoing preferred embodiments wherein the method reduces the incidence of HZ by at least 95%.

Also particularly preferred are the foregoing preferred embodiments wherein the method reduces the incidence of PHN by at least 85%. More particularly preferred are the foregoing preferred embodiments wherein the method reduces the incidence of PHN by at least 87%.

Even more preferred are each of the foregoing preferred and particularly preferred embodiments wherein the VZV gE antigen has the sequence of SEQ ID No. 1 and is present in a dose of 50 μg, and wherein QS21 and 3D-MPL are also present in a dose of 50 rig.

A further particular embodiment is an immunogenic, e.g. vaccine, composition comprising a VZV gE antigen truncated to remove the carboxy terminal anchor region or derivative thereof, in combination with an adjuvant comprising a QS21, a 3D-MPL and liposomes comprising cholesterol for use in a method for protecting against or preventing herpes zoster (HZ) and/or post herpetic neuralgia in individuals 80 years of age or older reducing the incidence of PHN by at least 60% or at least 70%, for at least 5 years.

The present invention is illustrated by the following, non-limiting examples.

### Example 1—Vaccine Efficacy Against HZ in Adults Aged 50 Years and Older

Example 1 describes the results of a phase III, randomized, observer-blind, placebo-controlled, multicentre, clinical vaccination trial demonstrating the prophylactic efficacy, safety, and immunogenicity of a candidate HZ vaccine, i.e. GSK Biologicals' VZV gE/AS01B vaccine, when administered intramuscularly on a 0, 2-month schedule in adults aged 50 years and older.

The study population includes males and females without severely immune-compromising conditions in the age ranges 50-59 years of age (YOA), 60-69 YOA, 70-79 YOA and ≧80 YOA. The 70-79 YOA and ≧80 YOA strata were combined for primary analyses. Apportionment of approximately 20-25% of the ≧70 YOA cohort to persons ≧80 YOA ensured that this particularly vulnerable population is adequately represented.

The candidate HZ vaccine tested in this trial is an adjuvanted recombinant VZV gE vaccine as described herein. A saline solution is included as a negative control (placebo) in this study to evaluate the efficacy and safety profile of the candidate HZ vaccine.

The objectives of the clinical vaccination trial included evaluation of vaccine efficacy in the prevention of HZ compared to placebo in subjects within each of the following age ranges: 50-59 YOA, 60-69 YOA and 70 YOA, as measured by the reduction in HZ risk.

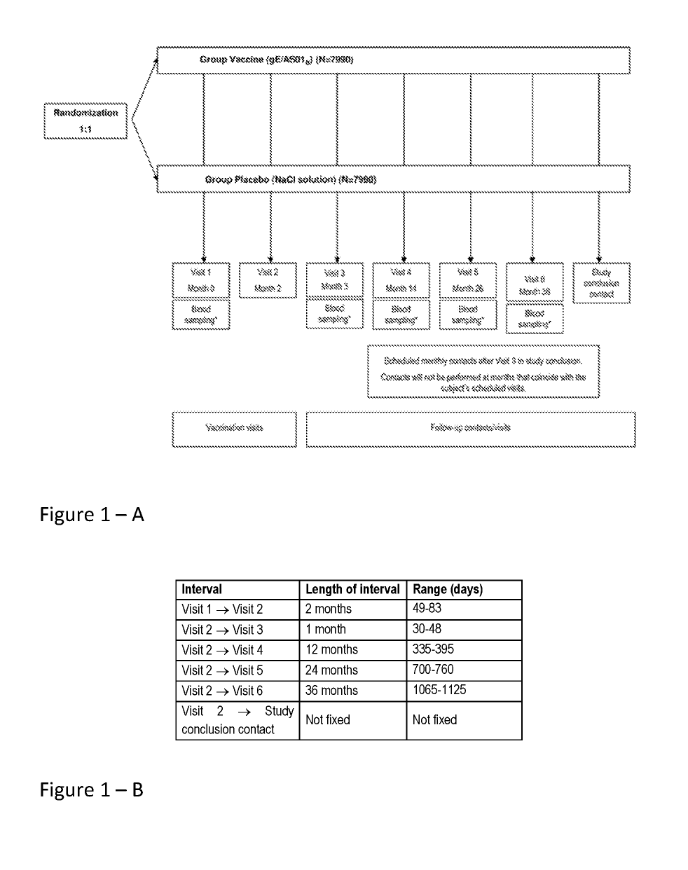

The study design is illustrated by FIG. 1.

The study encompassed two treatment groups, a placebo group and a vaccine group. The placebo group received NaCl solution as a control. The NaCl solution was provided in monodose vials (0.5 mL/dose) containing 150 mM NaCl per 0.5 mL dose. The vaccine group received the study vaccine. Each 0.5 mL dose of study vaccine contained 50 μg of VZV gE antigen, 50 μg of 3D-MPL, 50 jag of QS21, and liposomes (DOPC+cholesterol). The study vaccine was supplied in 2 vials, one containing the VZV gE antigen, and the other containing Adjuvant System AS01B.
The AS01B Adjuvant System is provided as a liquid formulation in monodose vials, each vial containing at least 0.5 mL of adjuvant. One 0.5 mL dose of AS01B formulation contains 50 μg of 3D-MPL and 50 μg of QS21 mixed with liposomes. The adjuvant system was formulated according to the method of preparation disclosed in example 3 and 4 of WO 2013/041572.
The VZV gE antigen was truncated gE having the sequence of SEQ ID no. 1. The antigen was obtained according to the method described in Example 2 of WO2006/094756, incorporated herein by reference in its entirety. The VZV gE antigen was provided in a lyophilized form in monodose vials. Each vial contained 62.5 μg of recombinant purified gE and formulation excipients. Therefore, when the 62.5 μg of VZV gE in each vial was reconstituted with the full volume of the AS01B adjuvant, each vaccine dose contained 50 μg of the VZV gE antigen per 0.5 mL dose of reconstituted vaccine.

The vaccination schedule was two doses of study vaccine or control saline for vaccine group and placebo group respectively, with the first dose at month 0 (visit 1) and second dose at month 2 (visit 2). The vaccine was administered intramuscularly.

Eligible subjects were randomized to vaccine/placebo group according to a 1:1 ratio (vaccine:placebo). Subjects were stratified by age: 50-59 YOA; 60-69 YOA; 70-79 YOA and 80 YOA in approximately an 8:5:3:1 ratio. The 70-79 YOA and 80 YOA strata were combined for primary analyses.

Primary HZ efficacy analysis occurred when the following condition was met: at least 196 confirmed HZ cases are accrued in the modified Total Vaccinated cohort (mTVc). The Total Vaccinated cohort (TVc) includes all vaccinated subjects with respect to the vaccine actually administered. The mTVc is the primary cohort for analysis of efficacy which excludes subjects in the TVc for efficacy analysis who were not administered with the second vaccination or who develop a confirmed case of HZ prior to 1 month after the second vaccination.

Table 1 lists the numbers of subjects in the mTVc included in the primary analysis.

| | Vaccine group | |
|---|---|---|
| | (n (%)) | Placebo group (n (%)) |
| Age | | |
| 50+ | 7344 (100.0) | 7415 (100.0) |
| 50-59 | 3492 (47.5) | 3525 (47.5) |
| 60-69 | 2141 (29.2) | 2166 (29.2) |
| ≧70 | 1711 (23.3) | 1724 (23.3) |
| ≧60 | 3852 (52.5) | 3890 (52.5) |
| Sex | | |
| Male | 2861 (39.0) | 2871 (38.7) |
| Female | 4483 (61.0) | 4544 (61.3) |
| Region | | |
| Australasia | 1555 (21.2) | 1574 (21.2) |
| Europe | 3786 (51.6) | 3828 (51.6) |
| Latin America | 711 (9.7) | 725 (9.8) |
| North America | 1292 (17.6) | 1288 (17.4) |

A suspected case of HZ was defined as new unilateral rash with pain (broadly defined to include allodynia, pruritis, or other sensations) and no alternative diagnosis. Suspected cases of HZ were confirmed in two ways:

1. By Polymerase Chain Reaction (PCR):

Rash lesion samples were collected from subjects clinically diagnosed as having a suspected case of HZ. The samples were transferred to GSK Biologicals or a validated laboratory designated by GSK Biologicals using standardised and validated procedures for laboratory diagnosis of HZ by PCR. Rash lesion samples were subjected to quantitative PCR (Q-PCR) targeting ORF62 (Mols J F et al. Sampling of herpes zoster skin lesion types and the impact on viral DNA detection. Journal of Virological Methods 2013; 188:145-7). If Q-PCR was positive for VZV, the suspected HZ was confirmed. If the Q-PCR was negative for VZV and positive for beta-actin, the case was classified as not HZ.

2. By the HZ Ascertainment Committee:

All suspected HZ cases were referred to or reviewed by the HZ Ascertainment Committee (HZAC), i.e. a panel of medical experts (blinded to Q-PCR results). The HZAC classified all referred cases as either “HZ” or “not HZ”. However, the HZAC classification served as the final case definition only when the case could not be confirmed or excluded by PCR, e.g., when all samples from a given subject were inadequate (negative Q-PCR for both VZV and beta-actin), or when no samples were available for a given subject. Therefore, definitive PCR results, when available, determined the final HZ case assignment. In such cases, the HZAC classification did not contribute to HZ case determination decision.

The HZAC consisted of three to five physicians with HZ expertise. HZAC members, participating as investigator in this study, did not evaluate cases from their own study site. HZAC members were blinded to treatment assignments. For every such case, each reviewing HZAC member was asked to make a clinical determination of whether the case is HZ based on review of the available clinical information (e.g., summary of the rash and pain evaluations, digital photographs of the subject's rash, and clinical progress notes). A suspected case of HZ was considered as “HZ” if the HZAC members concurred unanimously; otherwise, it was classified as “not HZ”. As described above, the HZAC case assignment was only considered as the final case assignment if definitive PCR results were not available.

Table 2 lists the outcome of the Primary HZ efficacy analysis

| | Vaccine group | | | Placebo group | | | Vaccine Efficacy | | | |
|---|---|---|---|---|---|---|---|---|---|---|
| | | | n/T | | | n/T | | 95% CI | | |
| Age strata | N | n | (per 1000) | N | n | (per 1000) | (%) | LL | UL | p-value |
| 50-59 | 3492 | 3 | 0.3 | 3525 | 87 | 7.8 | 96.57 | 89.62 | 99.31 | <0.0001 |
| 60-69 | 2141 | 2 | 0.3 | 2166 | 75 | 10.8 | 97.36 | 90.14 | 99.69 | <0.0001 |
| ≧70 | 1711 | 1 | 0.2 | 1724 | 48 | 9.4 | 97.93 | 87.91 | 99.95 | <0.0001 |
| ≧60 | 3852 | 3 | 0.2 | 3890 | 123 | 10.2 | 97.58 | 92.77 | 99.51 | <0.0001 |
| ≧50 | 7344 | 6 | 0.3 | 7415 | 210 | 9.1 | 97.16 | 93.72 | 98.97 | <0.0001 |

N = number of subjects included in each group
n = number of subjects having at least one herpes zoster (HZ) confirmed case
n/T (per 1000) = Incidence rate of subjects reporting at least one event per year
LL, UL = 95% Lower and Upper confidence limits
CI, Confidence Interval

### Example 2—Vaccine Efficacy Against HZ in Immuno-Compromised Adults

Example 2 describes a phase III, randomised, observer-blind, placebo-controlled, multicentre, clinical trial demonstrating the prophylactic efficacy, safety, and immunogenicity of an adjuvanted VZV gE candidate vaccine when administered intramuscularly on a two-dose schedule to adults of all ages with compromised immune systems, in casu adult autologous haematopoietic stem cell transplant (HCT) recipients.

The adjuvanted VZV gE candidate vaccine being tested in this trial is the same candidate vaccine tested in the trial described in Example 1. Since the candidate vaccine is a subunit vaccine, there is no risk that the vaccine itself will cause varicella or HZ, a potential concern following vaccination with a live VZV vaccine.

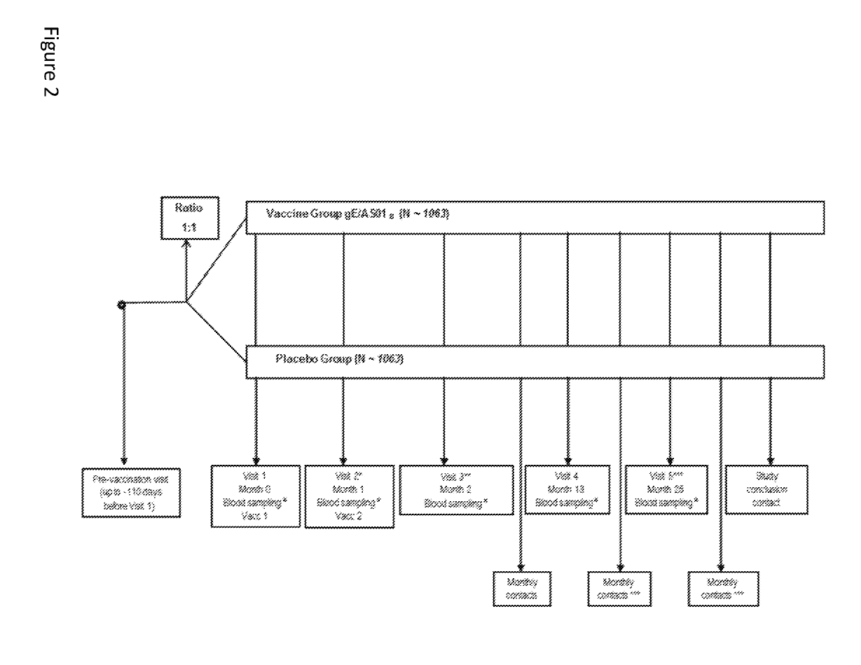

The study design is illustrated by FIG. 2.

The primary objective of the clinical trial is to evaluate vaccine efficacy (VE) in the prevention of HZ in autologous HCT recipients 18 years of age and older. Further objectives include VE in reducing the total duration of ‘worst’ HZ-associated pain over the entire pain reporting period in autologous HCT recipients 18 years of age and older with confirmed HZ, VE in the reduction of confirmed HZ-associated complications in autologous HCT recipients 18 years of age and older, VE in the prevention of PHN in autologous HCT recipients 18 years of age and older, VE in the prevention of PHN in autologous HCT recipients 18 years of age and older with confirmed HZ, etc.

### Example 3—Vaccine Efficacy Against HZ in Adults Aged 70 Years and Older

Example 3 describes the results of a phase III, randomized, observer-blind, placebo-controlled, multicentre, clinical vaccination trial assessing the prophylactic efficacy, safety, and immunogenicity of a candidate HZ vaccine, i.e. GSK Biologicals' VZV gE/AS01B vaccine, when administered intramuscularly on a 0, 2-month schedule in adults aged 70 years and older. Said trial has been conducted concurrently to the phase III trial described in Example 1.

The study population included males and females without severely immune-compromising conditions in the age ranges of 70-79 years of age (YOA) and 80 YOA. Apportionment of approximately 20-25% of the 70 YOA cohort to persons 80 YOA ensured that this particularly vulnerable population was adequately represented.

The candidate HZ vaccine tested in this trial was an adjuvanted recombinant VZV gE vaccine as described herein. A saline solution was included as a negative control (placebo) in this study to evaluate the efficacy and safety profile of the candidate HZ vaccine.

The objectives of the clinical vaccination trial included evaluation of vaccine efficacy in the prevention of HZ compared to placebo in subjects ≧70 YOA, as measured by the reduction in HZ risk.

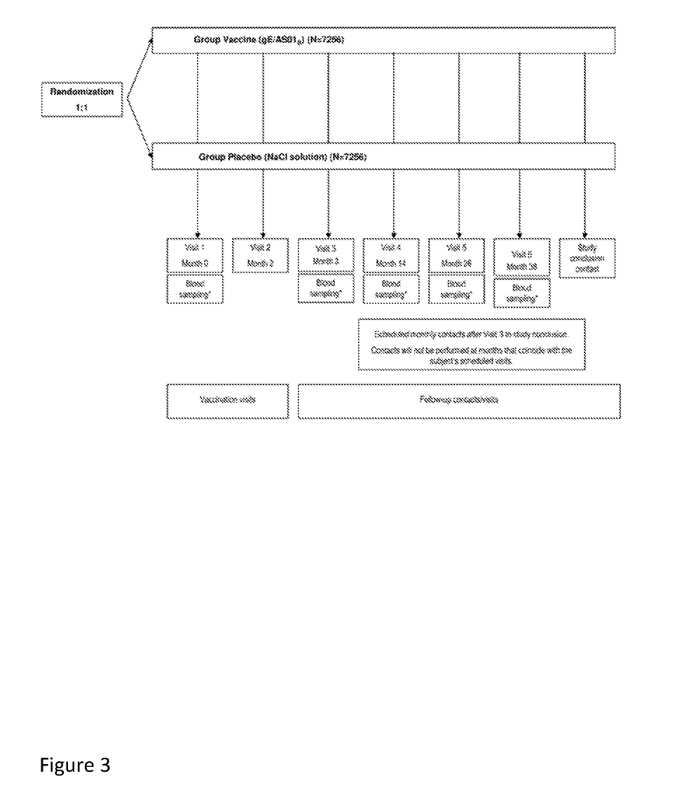

The study design is illustrated by FIG. 3.

The study encompassed two treatment groups, a placebo group and a vaccine group. The placebo group received NaCl solution as a control. The NaCl solution was provided in monodose vials (0.5 mL/dose) containing 150 mM NaCl per 0.5 mL dose. The vaccine group received the study vaccine. Each 0.5 mL dose of study vaccine contained 50 μg of VZV gE antigen, 50 μg of 3D-MPL, 50 jag of QS21, and liposomes (DOPC+cholesterol). The study vaccine was supplied in 2 vials, one containing the VZV gE antigen, and the other containing Adjuvant System AS01B. The AS01B adjuvant and the VZV gE antigen are as described in Example 1.

Eligible subjects were randomized to vaccine/placebo group according to a 1:1 ratio (vaccine:placebo). Subjects were stratified by age: 70-79 YOA and ≧80 YOA in approximately a 3:1 ratio. The 70-79 YOA and 80 YOA strata were combined for primary analyses.

Based on the efficacy results obtained in the trial described in Example 1, the statistical power of vaccine efficacy in the prevention of HZ in the present trial has been re-evaluated, and as a result, primary HZ efficacy analysis was re-evaluated to occur when the following condition was met: at least 211 confirmed HZ cases were accrued in the modified Total Vaccinated cohort (mTVc). The Total Vaccinated cohort (TVc) includes all vaccinated subjects with respect to the vaccine actually administered. The mTVc is the primary cohort for analysis of efficacy which excludes subjects who were not administered with the second dose of vaccine or placebo or who developed a confirmed case of HZ prior to a month after the second dose.

Table 3 lists the numbers of subjects in the mTVc included in the primary analysis

| | Vaccine group | |
|---|---|---|
| | (n (%)) | Placebo group (n (%)) |
| Age | | |
| 70-79 | 5114 (78.2) | 5189 (78.4) |
| ≧80 | 1427 (21.8) | 1433 (21.6) |
| Sex | | |
| Male | 2977 (45.5) | 2986 (45.1.9) |
| Female | 3564 (54.5) | 3636 (54.9) |
| Region | | |
| Australasia | 1211 (18.5) | 1240 (18.7) |
| Europe | 3567 (54.5) | 3604 (54.4) |
| Latin America | 485 (7.4) | 493 (7.4) |
| North America | 1278 (19.5) | 1285 (19.4) |

A suspected case of HZ was defined as described in Example 1 and confirmed in two ways as also described in Example 1.

Table 4 lists the outcome of the Primary HZ efficacy analysis

| | Vaccine group | | | Placebo group | | | Vaccine Efficacy | | | |
|---|---|---|---|---|---|---|---|---|---|---|
| | | | n/T | | | n/T | | 95% CI | | |
| Age strata | N | n | (per 1000) | N | n | (per 1000) | (%) | LL | UL | p-value |
| 70-79 | 5114 | 17 | 0.9 | 5189 | 169 | 8.8 | 90.02 | 83.54 | 94.32 | <0.0001 |
| ≧80 | 1427 | 6 | 1.2 | 1433 | 54 | 11.0 | 89.08 | 74.65 | 96.16 | <0.0001 |
| Overall ≧70 | 6541 | 23 | 0.9 | 6622 | 223 | 9.2 | 89.79 | 84.29 | 93.66 | <0.0001 |

N = number of subjects included in each group
n = number of subjects having at least one herpes zoster (HZ) confirmed case
n/T (per 1000) = Incidence rate of subjects reporting at least one event per year
LL, UL = 95% Lower and Upper confidence limits
CI, Confidence Interval

### Example 4—Pooled Trials: Vaccine Efficacy Against HZ and Vaccine Efficacy Against PHN

As mentioned earlier, the phase III trial described in Example 1 and the phase III trial described in Example 3 have been conducted concurrently to evaluate efficacy of GSK Biologicals' VZV gE/AS01B vaccine. As described in Example 1 and Example 3, in each phase III trial, vaccine efficacy in the prevention of HZ was determined separately and independently as a primary endpoint.

PHN is a known complication of HZ, which has a higher incidence in the population of individuals aged 70 and older. Because the vaccine efficacy reached against HZ, as obtained in the phase III trial described in Example 1 (see Table 2) was so high, and no breakthrough cases of PHN occurred in the this trial, the primary analysis for vaccine efficacy against PHN was carried out as a pooled analysis of both trials. Such pooling of the data obtained in each of the trials allowed to maximize the statistical power of said analysis, and accrue sufficient PHN cases. Pooling of data from the two trials is justified based on a similarity in study design, including treatment groups, randomization ratio, inclusion and exclusion criteria (apart from the age of the enrolled subjects), subject evaluations, case definitions, and definition of cohorts for analysis.

Accordingly, the objectives of pooling were two-fold. For the objective of preventing HZ that had already been demonstrated in Example 1 and Example 3 (see Table 2 and Table 4, respectively), the pooled analyses provided more robust estimates of vaccine efficacy with a smaller confidence interval (CI) for efficacy endpoints. For the objective of preventing PHN, the pooled analyses provided the highest power to generate statistically significant results. Therefore, the co-primary objectives of the pooling was the evaluation of vaccine efficacy compared to placebo (i) in the prevention of PHN in subjects 70 YOA across both phase III trials, as measured by the reduction in the occurrence of PHN, and (ii) in the prevention of HZ in subjects 70 YOA across both phase III trials, as measured by the reduction in the occurrence of HZ. As a secondary objective, vaccine efficacy against PHN in subjects in the age range of 50 YOA was also analyzed.

4.1 Vaccine Efficacy Against PHN in Adults Aged 50 Years and Older

Primary PHN efficacy analysis occurred when the following condition was met: at least 35 PHN cases in subjects ≧70 YOA were accrued in the modified Total Vaccinated cohort (mTVc), when pooling the two trials. The Total Vaccinated cohort (TVc) includes all vaccinated subjects with respect to the vaccine actually administered. The mTVc is the primary cohort for analysis of efficacy which excludes subjects who were not administered with the second dose of vaccine or placebo or who developed a confirmed case of HZ prior to a month after the second dose.

PHN was defined by the presence of HZ-associated severe “worst” pain persisting or appearing more than 90 days after onset of the HZ rash (confirmed case). Severe “worst” pain was defined as HZ-associated pain rated as 3 or greater on a scale from 0 to 10 according to the question of Zoster Brief Pain Inventory (ZBPI): “Please rate your pain by circling the one number that best describes your pain at its worst in the last 24 hours” (Coplan et al. 2004. J. Pain. 5(6):344-356).

Table 5 lists the outcome of the Primary PHN efficacy pooled analysis

| | Vaccine group | | | Placebo group | | | Vaccine Efficacy | | | |
|---|---|---|---|---|---|---|---|---|---|---|
| | | | n/T | | | n/T | | 95% CI | | |
| Age strata | N | n | (per 1000) | N | n | (per 1000) | (%) | LL | UL | p-value |
| ≧70 | 8250 | 4 | 0.1 | 8346 | 36 | 1.2 | 88.78 | 68.70 | 97.10 | <0.0001 |
| ≧50 | 13881 | 4 | 0.1 | 14035 | 46 | 0.9 | 91.22 | 75.95 | 97.70 | <0.0001 |
| ≧60 | 10390 | 4 | 0.1 | 10512 | 38 | 1.0 | 89.39 | 70.53 | 97.25 | <0.0001 |
| ≧80 | 1782 | 2 | 0.3 | 1792 | 7 | 1.1 | 71.16 | −51.51 | 97.08 | 0.1844 |

N = number of subjects included in each group
n = number of subjects having at least one PHN case
n/T (per 1000) = Incidence rate of subjects reporting at least one event per year
LL, UL = 95% Lower and Upper confidence limits
CI, Confidence Interval

4.2 Vaccine Efficacy Against HZ in Adults Aged 70 Years and Older

Table 6 lists the outcome of the Primary HZ efficacy pooled analysis

| | Vaccine group | | | Placebo group | | | Vaccine Efficacy | | | |
|---|---|---|---|---|---|---|---|---|---|---|
| | | | n/T | | | n/T | | 95% CI | | |
| Age strata | N | n | (per 1000) | N | n | (per 1000) | (%) | LL | UL | p-value |
| ≧80 | 1782 | 6 | 1.0 | 1792 | 68 | 11.1 | 91.37 | 80.22 | 96.94 | <0.0001 |
| 70-79 | 6468 | 19 | 0.8 | 6554 | 216 | 8.9 | 91.27 | 86.04 | 94.85 | <0.0001 |
| Overall ≧70 | 8250 | 25 | 0.8 | 8346 | 284 | 9.3 | 91.30 | 86.88 | 94.46 | <0.0001 |

N = number of subjects included in each group
n = number of subjects having at least one HZ confirmed case
n/T (per 1000) = Incidence rate of subjects reporting at least one event per year
LL, UL = 95% Lower and Upper confidence limits
CI, Confidence Interval

1-42. (canceled)

43. A method for preventing herpes zoster (HZ) in a human individual for at least 4 years, comprising the steps of administering to the individual an immunogenic composition comprising:
(a) a VZV gE antigen truncated to remove the carboxy terminal anchor region, and
(b) an adjuvant comprising a saponin, a TLR-4 agonist and liposomes.

44. A method for preventing herpes zoster in an individual comprising the steps of:
(a) selecting an individual from a population that is poorly protected by a live attenuated VZV composition, and,
(b) administering a first and a second dose of an immunogenic composition comprising a VZV gE antigen truncated to remove the carboxy terminal anchor region, and an adjuvant comprising a saponin, a TLR-4 agonist and liposomes,
wherein the prevention of HZ lasts for at least 4 years after administration of the second dose.

45. The method according to claim 44, wherein the method has an efficacy of reducing the occurrence of HZ by 60% or more in a vaccinated population.

46. The method according to claim 44, where said population consists of human individuals older than 70 years of age.

47. The method according to claim 44, where said population consists of immune-compromised human individuals.

48. The method according to claim 44, where Post Herpetic Neuralgia (PHN) is prevented in said individual for at least 4 years after administration of the second dose.

49. The method according to claim 48, wherein the method has an efficacy of reducing the occurrence of PHN by 70% or more in a vaccinated population.

50. The method according to claim 44, where said first and second dose are administered at an interval selected from 2, 6 and 12 months.

51. The method according to claim 44, where said first and second dose are administered at an interval of between 1 week and 12 months.

52. The method according to claim 44, wherein the VZV gE antigen is not in the form of a fusion protein.

53. The method according to claim 44, wherein the VZV gE antigen comprises the sequence of SEQ ID NO: 1.

54. The method according to claim 44, wherein the saponin is QS21.

55. The method according to claim 44, wherein the TLR-4 agonist is 3-O-desacyl-4′-Monophosphoryl Lipid A (3D-MPL).

56. The method according to claim 44, wherein the liposomes comprise a sterol.

57. A method for reducing the incidence of HZ in a population of human individuals, comprising administering to each individual a first and a second dose of an immunogenic composition comprising a VZV gE antigen truncated to remove the carboxy terminal anchor region and an adjuvant comprising a saponin, a TLR-4 agonist and liposomes, wherein the occurrence of HZ is reduced by 60% or more in said population.

58. The method according to claim 57, where said population consists of individuals older than 70 years of age.

59. The method according to claim 57, where said population consists of immune-compromised individuals.

60. The method according to claim 57, where said first and second dose are administered at an interval selected from 2, 6 and 12 months.

61. The method according to claim 57, where said first and second dose are administered at an interval of between 1 week and 12 months.

62. The method according to claim 57, wherein the method further reduces the occurrence of PHN by 70% or more in said population.
